(19) United States
(12) Patent Application Publication
(10) Pub. No.: US 2017/0156455 A1
(43) Pub. Date: Jun. 8, 2017

(54) BRACELET OR STRAP

(71) Applicant: The Swatch Group Research and Development Ltd, Marin (CH)

(72) Inventor: Nicolas FRANCOIS, Neuchatel (CH)

(73) Assignee: The Swatch Group Research and Development Ltd, Marin (CH)

(21) Appl. No.: 15/371,977

(22) Filed: Dec. 7, 2016

(30) Foreign Application Priority Data
Dec. 8, 2015 (EP) 15198534.8

(51) Int. Cl. A44C 5/00; A01N 59/06; A01N 55/02; A01N 25/08; A61Q 19/00; A61K 8/97; A61K 8/92; A61K 8/11; A61K 8/73; A01N 59/26; A61K 8/64

(52) U.S. Cl. CPC A44C 5/0023; A01N 59/26; A01N 59/06; A01N 55/02; A01N 25/08; A61K 8/64; A61K 8/97; A61K 8/92; A61K 8/11; A61K 8/735; A61Q 19/00; A61K 2800/75; A61K 2800/805

(57) ABSTRACT

The invention relates to a bracelet or strap, in particular a watch band, comprising at least one organic material based portion intended to be placed in contact with the user's skin, said organic material containing at least one functional agent, such as an antibacterial agent. According to the invention, said organic material also contains a cosmetic agent for the skin, arranged to be able to be in contact with the user's skin when the bracelet is worn.

The invention also concerns methods for manufacturing such a bracelet or strap.

This application claims priority form European Patent Application No. 15198534.8 filed on Dec. 8, 2015, the entire disclosure of which is hereby incorporated herein by reference.

### FIELD OF THE INVENTION

The invention relates to a bracelet or strap, in particular a watch band, comprising at least one organic material based portion intended to be placed in contact with the user's skin, said organic material containing at least one functional agent, for example an antibacterial agent.

The present invention also concerns methods for the manufacture of said bracelet or strap.

### BACKGROUND OF THE INVENTION

Such straps comprising an antibacterial agent are known and are for example described in DE 20315119. According to this document, the leather watch strap comprises an inner band in contact with the user's skin, made from a leather treated to exhibit antibacterial properties. This strap has the advantage of not developing unpleasant odours, particularly with perspiration.

There are also known bracelets made of plastic materials and comprising active ingredients used in the form of microcapsules embedded in the bracelet material. In the publication KR 20080003342 the functional agent is a vitamin. In FR 2842107, the functional agent may be a perfume, or a dose of medication. The vitamin or medication are released by the bracelet rubbing against the skin and will diffuse in the organism to treat the wearer of the bracelet.

However, some active ingredients incorporated in the bracelet, such as antibacterial agents or perfumes, may become irritants for the skin, especially in the event of prolonged contact between the bracelet and the skin, and more particularly in hot and humid conditions. Indeed, such conditions promote maceration at the interface between the skin and the bracelet, which creates a very uncomfortable sensation for the watch wearer. Combined with the friction of the bracelet on the skin, wearing a bracelet in which active ingredients are incorporated may sooner or later result in skin irritation. It then becomes uncomfortable to wear the bracelet or even impossible in case of significant skin lesions.

### SUMMARY OF THE INVENTION

It is an object of the invention to overcome the various drawbacks of known bracelets.

More precisely, it is an object of the invention to provide a bracelet or strap which, although incorporating at least one functional agent, can eliminate the risk of skin irritation and provide greater wearing comfort.

To this end, the present invention concerns a bracelet or strap, in particular a watch band, comprising at least one organic material based portion intended to be placed in contact with the user's skin, said organic material containing at least one functional agent.

According to the invention, said organic material also contains at least one cosmetic agent for the skin, arranged to be able to be in contact with the user's skin when the bracelet is worn.

Thus, when the bracelet according to the invention is worn, the bracelet diffuses into the user's skin at least one cosmetic agent for skin, acting on the skin in contact with the bracelet to prevent any irritation of the skin. Wearing comfort is therefore greatly improved.

The present invention also concerns a method for manufacture of a bracelet or strap as defined above, in particular a watch band, comprising at least one organic material based portion intended to be placed in contact with the user's skin, said organic material containing at least one functional agent and at least one cosmetic agent for the skin arranged to be able to be in contact with the user's skin when the bracelet is worn, said method comprising the steps of:
a) preparing at least one cosmetic agent for skin in encapsulated form arranged to release said cosmetic agent through friction on the skin
b) producing a formulation using said organic material
c) adding to the formulation obtained in step b) at least one cosmetic agent for skin in the encapsulated form obtained in step a) and at least one functional agent
d) making the bracelet from the formulation obtained in step c).

The present invention also concerns a method for manufacture of a bracelet or strap as defined above, in particular a watch band, comprising at least one organic material based portion intended to be placed in contact with the user's skin, said organic material containing at least one functional agent and at least one cosmetic agent for the skin arranged to be able to be in contact with the user's skin when the bracelet is worn, said method comprising the steps of:
a′) taking an element intended to form the organic material based portion of the bracelet, said organic material having no cosmetic agent for skin, and possibly no functional agent
b′) incorporating at least one cosmetic agent for skin, and possibly at least one functional agent if the latter is not already present, in the organic material based portion of the bracelet in step a′) by a method of impregnation
c′) making the bracelet, said step c′) can be performed between step a′) and step b′) or after step b′).

### DETAILED DESCRIPTION OF PREFERRED EMBODIMENTS

The present invention concerns a bracelet or strap, in particular a watch band. This bracelet comprises at least one organic material based portion intended to be placed in contact with the user's skin. This portion may form one or more areas of the bracelet or the entire bracelet.

In the present description, the term “organic material” preferably refers to leather, elastomers, thermoplastic elastomers, or any other organic material used for making bracelets or straps, and particularly watch bands.

As regards elastomers, these are advantageously rubber-like vulcanizable elastomers. They may be made, for example, from a fluorocarboned elastomer, known to be pleasant to the touch, or a mixture of fluorocarboned elastomers of the following type: copolymers of tetrafluoroethylene/propylene with a molecular weight of 70,000 to 250,000 or of tetrafluoroethylene/perfluoro(methylvinylether) with a molecular weight of 100,000 to 300,000, or terpolymers of tetrafluoroethylene/propylene/vinylidene fluoride with a molecular weight of 100,000 to 250,000. Such elastomers are available on the market. The rubber elastomer may be mixed with other nitrile (NBR), hydrogenated nitrile (HNBR) or silicon (VMQ, FMVQ . . . ) elastomers.

The thermoplastic elastomer may be a thermoplastic elastomer of polyurethane type, or an ethylene vinyl acetate copolymer, or amide block copolymer. These thermoplastic elastomers are available on the market.

The organic material contains at least one functional agent.

In the present description, the term “functional agent” means a substance incorporated in the bracelet to perform a certain function, which may equally be therapeutic or non-therapeutic. Thus, the functional agent present in the bracelet may, for example, be a medication, a perfume, an antibacterial agent, a cooling agent such as menthyl lactate, and mixtures thereof, or any other suitable functional agent to give the bracelet the desired property. Depending on its function, the functional agent may be arranged to be able to be in contact with the user's skin when the bracelet is worn. Preferably, the functional agent present in the bracelet is an antibacterial agent that can be selected, for example, from the group comprising zinc pyrithione, silver phosphate, aluminium salts, such as aluminium chlorohydrate, and absorbent microspheres. Such an antibacterial agent may prevent the development of unpleasant odours essentially due to perspiration of the wearer of the bracelet. Aluminium chlorohydrate has the advantage of also being an antiperspirant agent. It is, of course, possible to combine different functional agents, such as a perfume and an antibacterial agent.

When the bracelet is made of leather, all the constituent elements of the bracelet can be treated with a functional agent, such as an antibacterial agent. Thus, the top of the bracelet, the lining, the stitching, the internal elements, such as padding or rip-stop elements, can also be treated with an antibacterial agent.

According to the present invention, the organic material of the portion of the bracelet intended to be placed in contact with the user's skin also contains at least one cosmetic agent for skin arranged to be able to be in contact with the user's skin when the bracelet is worn. In the present description, the term “cosmetic agent for skin” means a cosmetic agent for skin care, i.e. an active ingredient acting on the skin to protect, care for and/or repair the skin to maintain skin integrity. Preferably, the cosmetic agent for skin is different from the functional agent.

Advantageously, the cosmetic agent for skin may be a repair agent which is intended to have a long-lasting effect on the skin. This repair agent may be a ceramide or hyaluronic acid.

According to another advantageous aspect of the invention, the cosmetic agent for skin may be an anti-irritant agent, intended to have an immediate effect on the skin by calming irritation. The anti-irritant agent may be acetyl tetrapeptide-15 dispersed in mannitol and sodium citrate or an oat extract in a water/glycerin dispersion.

Preferably, a repair agent is used in combination with an anti-irritant agent.

In a particularly advantageous manner, the cosmetic agent for skin may be in encapsulated form arranged in order to release said cosmetic agent through friction on the skin when the bracelet is worn by the user. Techniques for encapsulation of principle ingredients are known to those skilled in the art and do not require a detailed description. It is, however, useful to specify that the cosmetic agent for skin is encapsulated in a resin that is sufficiently resistant to resist the shaping process. Such a resin is, for example, melamine.

The bracelet according to the invention may be obtained by different methods.

In a first variant, the method of the invention comprises the steps of:
a) preparing at least one cosmetic agent for skin in encapsulated form arranged to release said cosmetic agent through friction on the skin
b) producing a formulation using said organic material
c) adding to the formulation obtained in step b) at least one cosmetic agent for skin in the encapsulated form obtained in step a) and at least one functional agent
d) making the bracelet from the formulation obtained in step c).

This first variant applies more particularly when the organic material is chosen from rubber-like vulcanizable elastomers and thermoplastic elastomers.

At least one cosmetic agent for skin is prepared in accordance with known techniques for encapsulation of active ingredients. The size of the microcapsules is, for example, 50 μm.

The base formulation of said organic material is a formulation conventionally used to make bracelets and straps, and particularly watch bands, from rubber-like or thermoplastic elastomers.

For example, for rubber-like vulcanizable elastomers, this formulation may contain, in a known manner:
elastomer or a mixture of elastomers, in an amount corresponding to 100 parts of the formulation,
20 to 40 parts of a filler, said filler being for example silica, kaolin, TiO₂ if the bracelet is light coloured, or carbon black if the bracelet is dark coloured or black,
2 to 4 parts of a vulcanization accelerator, such as a manganese oxide,
4 to 6 parts of a vulcanization agent, such as peroxides,
3 to 7 parts of additives such as carnauba wax,
0.5 to 2 parts of additives for the dye system.
   It is clear that any other appropriate formulation may be used.

Once the formulation is prepared, at least one cosmetic agent for skin in micro-encapsulated form and at least one functional agent are added to said formulation in a compounding step. For example, the cosmetic agent content is comprised between 1 and 40 wt %, preferably between 2 and 20 wt %, with respect to the total weight of the formulation, and the functional agent content is comprised between 0.1 and 1 wt %, and preferably between 0.2 and 0.4 wt %, with respect to the total weight of the formulation.

The bracelet is then made by injection or compression of the obtained formulation comprising the cosmetic agent for skin and at least one functional agent. These manufacturing techniques are known and do not require a detailed description. The advantage of this method is that it permits better integration of the cosmetic agent for skin and the function agent in the organic material, and in particular for vulcanizable elastomers due to the vulcanization, which will limit problems of migration.

In a second variant, the method of the invention comprises the steps of:
a′) taking an element intended to form the organic material based portion of the bracelet, said organic material having no cosmetic agent for skin, and possibly no functional agent
b′) incorporating at least one cosmetic agent for skin, and possibly at least one functional agent if the latter is not already present, in the organic material based portion of the bracelet in step a′) by a method of impregnation
c′) making the bracelet, said step c′) can be performed between step a′) and step b′) or after step b′).

According to a first embodiment, the impregnation method is achieved by simple impregnation. This embodiment may be applied especially when the element intended to form the organic material based portion is made of leather. Preferably, this element is the bracelet or strap lining, which is advantageously pre-treated with at least one functional agent, such as an antibacterial agent.

According to this first embodiment, step a′) is a dyeing step or a leather nourishing step, more particularly of the bracelet lining that already contains the antibacterial agent, and impregnation step b′) is performed jointly with step a′), at least one cosmetic agent for skin having been pre-prepared in encapsulated or emulsion form. Preferably, the cosmetic agent for skin is encapsulated in a polymer to resist the temperature and pH conditions required by the dyeing or leather nourishing step. Melamine can be used for example.

According to a second embodiment the impregnation method is a supercritical fluid impregnation method. Such an impregnation method may comprise the following steps:
dissolving at least one cosmetic agent for skin and at least one functional agent if the latter is not already present in the bracelet, in a support liquid to form a solution; the functional agent is used in contents of 0.5 to 10 wt %, and preferably in a content of 2.5 wt % if the functional agent is an antibacterial agent, and the cosmetic agent for skin is preferably prepared in the form of an emulsion (glycerin/water) preferably comprising between 4 and 10 wt % of cosmetic agent.
placing the element intended to form the organic material based portion under ambient pressure in a pressurization chamber;
hermetically sealing the chamber;
impregnating the element intended to form the organic material based portion with the solution by means of a fluid in supercritical conditions or close to supercritical conditions in the chamber at a pressure comprised between 3 MPa and 6 MPa, at a temperature comprised between 25° C. and 65° C., preferably equal to 35° C., for a duration comprised between 1 min and 12 mins, preferably equal to 10 mins;
releasing the pressure in the chamber so that the support liquid diffuses out of the element intended to form the organic material based portion and in order to trap the cosmetic agent and possibly the functional agent inside the element intended to form the organic material based portion.

Advantageously, the supercritical fluid is carbon dioxide and the support liquid is chosen from hexane, isopropanol or d-limonene. Preferably the impregnation is achieved at a constant flow rate.

Advantageously, there is then provided a step of eliminating residues which may be achieved by washing with clean water or by wiping the element intended to form the impregnated organic material based portion.

When the element intended to form the organic material based portion of the bracelet is chosen from leather, elastomers, and thermoplastic elastomers, step b′) of supercritical fluid impregnation may be performed after step c′), i.e. on an already formed bracelet. More particularly, when the organic material is an elastomer or a thermoplastic elastomer, the method for supercritical fluid impregnation with the cosmetic agent for skin and functional agent is performed on the already formed bracelet by injection or by compression from an elastomer formulation containing no cosmetic agent for skin or functional agent. When the organic material is leather, the method for supercritical fluid impregnation with cosmetic agent for skin is performed on the leather bracelet in the finished state and already treated with a functional agent such as an antibacterial agent.

According to another possibility, when the element intended to form the organic material based portion is leather, step a′) is a leather cutting step, and supercritical fluid impregnation step b′) is performed close to step a′) and before step c′). More specifically, supercritical fluid impregnation step b′) is performed after the leather cutting step. The leather bracelet is then finished.

The bracelet or strap according to the invention can offer the properties provided by at least one functional agent, particularly the antibacterial agent, while protecting the user's skin, as a result of the diffusion of at least one cosmetic agent for skin at the point of contact between the bracelet and the skin of the wearer of the bracelet. Thus, wearing comfort is greatly improved.

Of course, the present invention is not limited to the illustrated examples and is capable of various variants and modifications that will be evident to those skilled in the art.

1. A bracelet or strap, comprising at least one organic material, said organic material containing at least one functional agent, wherein said organic material also contains at least one cosmetic agent for skin arranged to be able to be in contact with the user's skin when the bracelet or strap is worn.

2. The bracelet or strap according to claim 1, wherein the organic material is chosen from the group consisting of leather, elastomers, and thermoplastic elastomers.

3. The bracelet or strap according to claim 1, wherein the functional agent is chosen from the group consisting of antibacterial agents, perfumes, cooling agents and mixtures thereof.

4. The bracelet or strap according to claim 3, wherein the antibacterial agent is chosen from the group consisting of zinc pyrithione, silver phosphate and aluminium salts.

5. The bracelet or strap according to claim 1, wherein the cosmetic agent for skin is chosen from the group consisting of a repair agent, an anti-irritant agent and mixtures thereof.

6. The bracelet or strap according to claim 5, wherein the repair agent is chosen from the group consisting of ceramides and hyaluronic acid.

7. The bracelet or strap according to claim 5, wherein the anti-irritant agent is chosen from the group consisting of acetyl tetrapeptide-15 and oat extract.

8. The bracelet or strap according to claim 1, wherein the cosmetic agent for skin is in encapsulated form arranged to release said cosmetic agent through friction on the skin.

9. A method for manufacture of a bracelet or strap, comprising at least one organic material, said organic material containing at least one functional agent and at least one cosmetic agent for the skin arranged to be able to be in contact with the user's skin when the bracelet or strap is worn, the method comprising:
a) preparing at least one cosmetic agent for skin in encapsulated form arranged to release said cosmetic agent through friction on the skin;
b) producing a formulation using said organic material;
c) adding to the formulation obtained in b) at least one cosmetic agent for skin in the encapsulated form obtained in a) and at least one functional agent; and
d) making the bracelet or strap from the formulation obtained in c).

10. The method according to claim 9, wherein the organic material is chosen from the group consisting of leather, elastomers, and thermoplastic elastomers.

11. The method for manufacture of a bracelet or strap, comprising at least one organic material, said organic material containing at least one functional agent and at least one cosmetic agent for the skin arranged to be able to be in contact with the user's skin when the bracelet or strap is worn, method comprising:
a′) taking an element intended to form the organic material based portion of the bracelet or strap, said organic material having no cosmetic agent for skin, and optionally no functional agent.
b′) incorporating at least one cosmetic agent for skin, and optionally at least one functional agent if the latter is not already present, in the organic material based portion of the bracelet or strap in a′) by a method of impregnation; and
c′) making the bracelet or strap, said making c′) being performed between a′) and b′) or after b′).

12. The method according to claim 11, wherein the impregnation method is a supercritical fluid impregnation method.

13. The method according to claim 12, wherein the supercritical fluid impregnation b′) is performed after c′).

14. The method according to claim 12, wherein the element intended to form the organic material based portion of the bracelet or strap is chosen from the group consisting of leather, elastomers and thermoplastic elastomers.

15. The method according to claim 12, wherein the element intended to form the organic material based portion is leather, and wherein a′) is a leather cutting step, and supercritical fluid impregnation b′) is performed close to a′) and before c′).

16. The method according to claim 11, wherein the element intended to form the organic material based portion is leather, wherein a′) is a dyeing step or a leather nourishing step, and wherein the impregnation b′) is performed jointly with a′), the cosmetic agent for skin having been pre-prepared in an encapsulated form.

17. The method according to claim 9, wherein the functional agent is chosen from the group consisting of antibacterial agents, perfumes, cooling agents and mixtures thereof.

18. The method according to claim 17, wherein the antibacterial agent is chosen from the group consisting of zinc pyrithione, silver phosphate and aluminium salts.

19. The method according to claim 9, wherein the cosmetic agent for skin is chosen from the group consisting of a repair agent, an anti-irritant agent and mixtures thereof.

20. The method according to claim 19, wherein the repair agent is chosen from the group consisting of ceramides and hyaluronic acid.

21. The method according to claim 19, wherein the anti-irritant agent is chosen from the group consisting of acetyl tetrapeptide-15 and oat extract.

22. The method according to claim 11, wherein the functional agent is chosen from the group consisting of antibacterial agents, perfumes, cooling agents and mixtures thereof.

23. The method according to claim 22, wherein the antibacterial agent is chosen from the group consisting of zinc pyrithione, silver phosphate and aluminium salts.

24. The method according to claim 11, wherein the cosmetic agent for skin is chosen from the group consisting of a repair agent, an anti-irritant agent and mixtures thereof.

25. The method according to claim 24, wherein the repair agent is chosen from the group consisting of ceramides and hyaluronic acid.

26. The method according to claim 24, wherein the anti-irritant agent is chosen from the group consisting of acetyl tetrapeptide-15 and oat extract.
